(19) United States
(12) Patent Application Publication
(10) Pub. No.: US 2026/0306802 A1
(43) Pub. Date: Oct. 8, 2026

(54) Elongate Medical Instrument With Twisted Core Wire

(71) Applicant: EPflex Feinwerktechnik GmbH, Dettingen/Erms (DE)

(72) Inventors: Senta SCHAUER, Reutlingen (DE); Bernhard UIHLEIN, Dettingen (DE)

(21) Appl. No.: 19/476,233

(22) PCT Filed: Apr. 18, 2024
(86) PCT No.: PCT/EP2024/060501

§ 371 Date: Oct. 17, 2025

(30) Foreign Application Priority Data
Apr. 19, 2023 (DE) 10 2023 109 898.5

(51) Int. Cl. A61M 25/09; A61B 17/00

(52) U.S. Cl. CPC A61M 25/09; A61B 17/00234; A61B 2017/00022; A61B 2017/00358; A61M 2025/09075; A61M 2025/09083; A61M 2025/0915; A61M 2025/09175; A61M 2025/09191

(57) ABSTRACT

An elongate medical instrument has a core wire extending from a proximal region of the instrument to a distal region of the instrument, which is formed from a wire material twisted around its longitudinal axis. The instrument has a conductor element arrangement which contains at least one electrically-conducting and/or light-conducting and/or fluid-conducting conductor element, that extends from the proximal region of the instrument to the distal region of the instrument in a helical manner synchronous with the twisting of the core wire. The instrument may be used, in particular in medical endoscopy, for guide wires and snare-wire instruments.

### BACKGROUND AND SUMMARY

The invention relates to an elongate medical instrument comprising a core wire extending from a proximal region of the instrument to a distal region of the instrument and formed from a wire material which is twisted around its longitudinal axis.

Elongate medical instruments are known in a wide variety of embodiments for different applications in medical technology, primarily in endoscopy, in particular in connection with catheter instruments. These include, for example, guide wires and snare instruments. The proximal region represents a rear region of the instrument, which can typically be coupled to a user-operable operator control unit. The distal region represents a front region of the instrument, with which the instrument can be introduced typically into a human or animal tissue channel in front of it and in which a functional element, for example a sensor element or a light source or a scissors, pliers or cutting tool, can be located at a distal end region of the instrument, depending on requirements. In many applications, it is also desirable for the distal end region of the instrument to have a lower flexural stiffness in at least one bending direction, alternatively in two bending directions or on all sides, than in the remaining region of the instrument. The region of the instrument between the proximal end region and the distal end region is commonly also referred to as the shaft region.

In the type of instruments under consideration at present, the core wire is formed from a wire material composed of metal or plastic and twisted around its longitudinal axis, i.e. the wire material is twisted around its own longitudinal axis which lies within the wire material itself, i.e. within its material cross section, in particular around its longitudinal center axis, so that, in the case of a flat-wire material for example, its side edges run in a helically wound manner. The twisting can be selected to be uniform, i.e. with a winding pitch of its side edges that remains constant over its twisted length, or different with a continuous or abrupt transition from one winding pitch to the other in different portions, depending on requirements.

Instruments of the kind mentioned at the outset are disclosed in the form of guide wires, in which the core wire is formed from a twisted flat wire material with a substantially rectangular cross section, in patent publications U.S. Pat. Nos. 5,313,967 and 9,492,642 B2.

Patent publication DE 10 2017 200 077 A1 discloses a medical guide wire with a helical spring piece composed of a flat wire material.

Laid-open publication EP 3 610 915 A1 discloses a guide wire having a proximal light source and a distal image sensor which is optically coupled to the light source via a light guide fiber which extends together with a core wire from a proximal region to a distal region of the guide wire. In this case, the optical fiber runs helically around the linearly extending core wire, or the core wire runs helically around the linearly extending optical fiber, or the core wire and the optical fiber both run helically around a longitudinal center axis of the guide wire in a twisted-together manner, the longitudinal center axis lying between the optical fiber material and the wire material of the core wire and thus outside the optical fiber material and the core wire material.

Laid-open publication US 2009/0112128 A1 discloses a guide wire which is made up of several wires which run, adjacently to one another around a longitudinal center axis of the guide wire, the longitudinal center axis being situated outside the longitudinal axes of the wires, leaving a central lumen between them, in a helically wound manner from a proximal region of the guide wire to a distal region of the guide wire and at least one of which is designed to be electrically conductive. In addition, one or more optical fibers can run in a wound manner from the proximal region to the distal region of the guide wire.

Snare instruments for medical endoscopy are used primarily for snaring foreign bodies, blood clots, stones, etc. in tissue channels of humans and animals. For this purpose, a snare basket, which is operated from a proximal operator control region via a pulling wire, in particular moved out of a sheathing such that it folds open and retracted back into such that it folds closed, is typically formed at the distal region of the instrument. Such a snare instrument is disclosed, for example, in laid-open publication WO 2015/139998 A1. In the present case, the core wire serves as a basket-operating pulling wire.

The invention is based on the technical problem of providing an elongate medical instrument of the kind mentioned at the outset which offers further functionalities and possible uses compared to the abovementioned prior art and can be manufactured with comparatively little outlay on production.

The invention solves this problem by providing an elongate medical specific instrument having features of the independent claim(s). Advantageous developments of the invention are the subject matter of the dependent claims, the wording of which is hereby made part of the description by reference. This in particular also includes all of the embodiments of the invention that arise from the combinations of features which are defined by the dependency references in the dependent claims.

The elongate medical instrument according to the invention comprises a core wire extending from a proximal region of the instrument to a distal region of the instrument and formed from a wire material which is twisted around its longitudinal axis, and further comprises at least one electrically conductive and/or light-guiding and/or fluid-conducting conductor element which extends from the proximal region of the instrument to the distal region of the instrument in a helical manner synchronously with the twisting of the core wire. Twisting the core wire offers advantages in terms of the bending behavior of the instrument, and using the twisted core wire offers advantages in terms of relatively low outlay on manufacturing. Owing to the presence of the conductor element, the instrument has correspondingly extended possible uses. The helical longitudinal profile of the conductor element is advantageously adapted to the twisted profile of the core wire.

The electrically conductive design of the conductor element can thus be used, for example, to transmit an electrical signal and/or electrical energy between the distal and the proximal region of the instrument, for example an electrical sensor signal of a sensor arranged in the distal instrument region to the proximal instrument region or electrical energy or an electrical control signal from the proximal instrument region to an electrical functional element arranged in the distal instrument region, such as an electrically actuable or operable scissors, pliers or cutting tool or an electrical light source.

In the light-guiding embodiment, the conductor element can be used, for example, to transmit an optical control signal from the proximal region to the distal region of the instrument, or to transmit a sensor signal from the distal region of the instrument to the proximal region, or to act as a light guide for a camera system.

In the fluid-conducting embodiment, the conductor element is of hollow design so as to form at least one lumen and can be used, for example, to supply a fluid from the proximal region to the distal region of the instrument or to discharge a fluid from the distal region of the instrument to the proximal region.

In a development of the invention, the wire material is a flat wire material with a polygonal cross section. The use of such a flat wire material is generally associated with comparatively low outlay on manufacturing and offers functional advantages for corresponding applications. Thus, in use, the friction between the twisted core wire and an inside of an element surrounding the instrument, such as a catheter tube, can be kept low since the frictional contact in this case is limited to the helically running side edges of the flat wire material and there is no greater, surface-area frictional contact. In alternative embodiments, a different type of wire material is used for the core wire, for example a round wire material, if this is sufficient or expedient for the application in question and/or offers manufacturing advantages.

In a development of the invention, the wire material has a ratio of wire width to wire thickness of between 1:1 and 4:1. The wire width is understood to mean the maximum cross-sectional extent and the wire thickness is understood to mean the minimum cross-sectional extent of the wire material. It has been found that a ratio of wire width to wire thickness for the twisted core wire in this range of between 1:1 and 4:1 is expedient for a very large number of applications of the instrument. In alternative embodiments, a wire material with a ratio of wire width to wire thickness greater than 4:1 can also be used.

In a development of the invention, the conductor element is inserted into an associated conductor receiving groove of the core wire. By insertion into the conductor receiving groove, if desired, an externally flush or non-protruding profile of the conductor element with the core wire can be achieved, i.e. the conductor element does not protrude radially beyond the core wire in this case. In alternative embodiments, the conductor element is arranged in a manner projecting radially from the outside of the core wire, the conductor element bearing with touching contact against the outside of the core wire or being at a certain, small distance from it, depending on the implementation.

In a development of the invention, the conductor element is arranged on a broad side of the core wire. This placement of the conductor element has the advantage that the lateral extent of the twisted core wire can be kept relatively small and can be limited, for example, to the wire width of the wire material used for the core wire. In alternative embodiments, the conductor element is arranged on a narrow side of the core wire, if this offers advantages for the application in question.

In a refinement of the invention, the conductor element arrangement contains a first conductor element on a first broad side of the core wire and a second conductor element on a second broad side of the core wire. With this measure, two conductor elements can be arranged on the core wire, without this having to increase the lateral dimension of the twisted core wire, which can then be determined by the wire width of the wire material used and thus the distance between opposite narrow sides. In other embodiments, one or more conductor elements can be arranged on one or more narrow sides of the core wire in addition or as an alternative to the conductor elements arranged on the broad side or sides of the core wire.

In a development of the invention, the core wire has at least one flat side surface and at least one rounded side surface. This implementation is expedient for applications in which the cross section of the core wire should have an at least partially rounded shape. In this case, it may be preferred to arrange the conductor element on the flat side surface of the core wire. In alternative embodiments, the core wire can have, for example, a polygonal cross section without rounded side surfaces or a circular or oval cross section without flat side surfaces, if this is expedient for corresponding applications.

In a development of the invention, the core wire and the conductor element arrangement are embedded in an embedding material from the proximal region of the instrument to the distal region of the instrument. The embedding material envelops the core wire and the conductor arrangement and secures these components of the instrument in their mutual position without any other measures having to be taken for this purpose. In alternative embodiments, such embedding of the core wire and the conductor element arrangement from the proximal instrument region to the distal instrument region is omitted, wherein the conductor element or elements can be held, for example, by an adhesive or welded connection on the core wire.

In a development of the invention, the instrument has a sheathing tube which surrounds the core wire and the conductor element arrangement from the proximal region of the instrument to the distal region of the instrument. The position of the conductor element or elements on the core wire can be secured by the sheathing tube, wherein the sheathing tube is preferably connected fixedly or at least rigidly in terms of axial movement to the core wire and further optionally also to the conductor element arrangement, i.e. the core wire and the conductor element arrangement are not axially movable relative to the sheathing tube in this case. In addition, the sheathing tube can fulfill other functions that are known per se, such as improving the sliding property of the instrument and/or electrically insulating the instrument radially to the outside. In alternative embodiments, the sheathing tube is omitted if this is not necessary for the application in question or does not have any advantages.

In a refinement of the invention, a fluid guiding lumen extending from the proximal region of the instrument to the distal region of the instrument is formed between the core wire and the sheathing tube. In this case, the sheathing tube additionally fulfills a fluid guiding function together with the core wire. In the fluid guiding volume, a fluid can be guided from the proximal instrument region to the distal instrument region or from the distal instrument region to the proximal instrument region. For applications of this kind, a wire material with a width/thickness ratio of more than 4:1 may be expedient, in order to have a relatively large cross section available for the fluid guiding lumen. In alternative embodiments, such a fluid-carrying cavity between the core wire and the sheathing tube is omitted, for example in cases in which an intermediate space between the core wire and the sheathing tube is filled with an embedding material.

In a development of the invention, the instrument comprises a stiffening wire arrangement which contains at least one stiffening wire which extends from the proximal region of the instrument to the distal region of the instrument in a helical manner synchronously with the twisting of the core wire. The stiffening wire arrangement can be used to provide the instrument with a desired bending behavior. The stiffening wire follows the twisting of the core wire, as a result of which production can be simplified and the lateral dimension of the instrument can be kept relatively small. In alternative embodiments, the instrument is provided without such a stiffening wire arrangement.

In a development of the invention, the core wire, in a distal tip region of the instrument, has a distal core wire portion conically tapered to a round cross section. An electrical functional element, to which the conductor element is guided, is arranged at a distal end of the instrument. The distal tip region of the instrument has a distal sheathing which surrounds the electrical functional element, the conically tapered distal core wire portion and the part of the conductor element running there. The conical tapering on the round cross section provides the distal core wire section with less bending stiffness, regardless of direction, than in the remaining, non-tapered portion. The conical tapering can be achieved, for example, by grinding. The electrical functional element at the distal tip region fulfills a corresponding function for the instrument, for example a sensor function or a wireless, electromagnetic control or tool function. The electronic functional element and the portion of the conductor element running in the distal tip region of the instrument are protected by the distal sheathing, which may be a seamlessly embedded sheathing or a sheathing surrounding in the manner of a tube. In alternative embodiments, the instrument is formed in the distal tip region in a different way, in a manner adapted to the respective application.

In a development of the invention, a sensor element is arranged in the distal region of the instrument, and the conductor element arrangement is led with a distal end to the sensor element. In this case, the conductor element arrangement can be used to supply the sensor element with energy or light or sensor commands and/or to conduct signals from the sensor element to the proximal region of the instrument.

Advantageous embodiments of the invention are illustrated in the drawings. These and further embodiments of the invention will be described in more detail below.

### BRIEF DESCRIPTION OF THE DRAWINGS

FIG. 1 shows a perspective side view of part of an elongate medical instrument with a twisted core wire composed of rectangular flat wire material and with a conductor element.

FIG. 2 shows a cross-sectional view along a line II-II in FIG. 1.

FIG. 3 shows the cross-sectional view of FIG. 2 for an instrument variant with two conductor elements.

FIG. 4 shows the side view of FIG. 1 for a variant with a twisted core wire composed of round wire material with a conductor receiving groove.

FIG. 5 shows the cross-sectional view of FIG. 2 for the variant of FIG. 4.

FIG. 6 shows the cross-sectional view of FIG. 5 for a variant with two conductor receiving grooves and conductor elements.

FIG. 7 shows a side view of part of a twisted core wire composed of rectangular flat wire material with a distal end region conically ground to a round cross section.

FIG. 8 shows the side view of FIG. 7 for a variant with an additional distal helical spring sheathing.

FIG. 9 shows the cross-sectional view of FIG. 3 for a variant with an additional sheathing tube.

FIG. 10 shows the cross-sectional view of FIG. 9 for a variant with a more flexible sheathing tube.

FIG. 11 shows the cross-sectional view of FIG. 9 for a variant with rounded core wire narrow sides.

FIG. 12 shows the cross-sectional view of FIG. 6 for a variant with an additional sheathing tube.

FIG. 13 shows the cross-sectional view of FIG. 9 for a variant with additional stiffening wire and/or additional conductor elements.

FIG. 14 shows the cross-sectional view of FIG. 13 for a variant with an embedded sheathing.

FIG. 15 shows a side view of part of a guide wire with the core wire of FIG. 7 and a distal electrical component.

FIG. 16 shows a side view of part of a guide wire with the core wire of FIG. 8, a sheathing tube and a fluid guiding lumen between the core wire and the sheathing tube.

FIG. 17 shows a view of a detail of a region XVII of FIG. 16.

FIG. 18 shows a partially sectioned side view of part of an elongate medical instrument in the form of a snare instrument with a twisted core wire according to FIG. 7 as a pulling wire, an existing conductor element arrangement not being shown, in a state with the snare basket folded-closed.

FIG. 19 shows the view of FIG. 18 in a state with the snare basket unfolded.

FIG. 20 shows a side view of the core wire and the conductor element arrangement of the snare instrument in a distal end region.

FIG. 21 shows a view of a detail of a region XIX in FIG. 18.

### DETAILED DESCRIPTION OF THE DRAWINGS

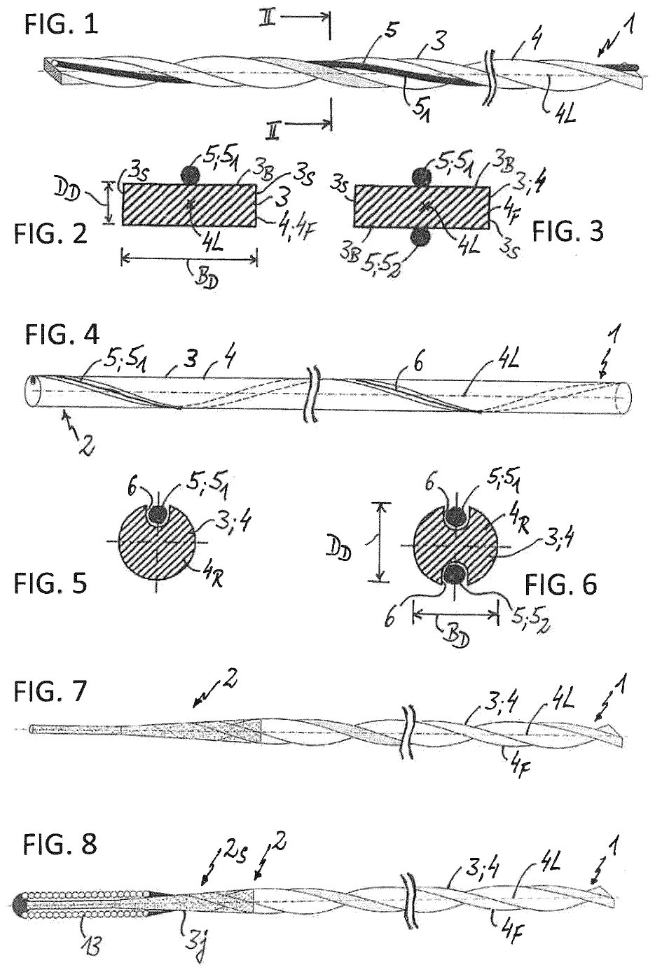

FIGS. 1 to 21 schematically show various advantageous embodiments of the elongate medical instrument according to the invention, which may be, in particular, a corresponding guide wire or a corresponding snare instrument, with the components of interest at present. As can be seen from these figures, the medical instrument contains a core wire 3 extending from a proximal region 1 to a distal region 2 of the instrument and formed from a wire material 4 that is twisted about its longitudinal axis 4L. More specifically, in the present case, this twisting is to be understood in such a way that the longitudinal axis 4L, about which the wire material 4 is twisted, is located in the wire material 4 itself, i.e. the wire material 4 is inherently twisted, in other words twisted about a longitudinal axis situated in its cross section. Furthermore, the instrument has a conductor element arrangement 5 which contains at least one electrically conductive and/or light-guiding and/or fluid-conducting conductor element 5₁ which extends from the proximal instrument region 1 to the distal instrument region 2 in a helically wound manner, the helical profile of the conductor element 5₁ being synchronous with the twisted profile of the core wire 3. In other words, the helical profile of the conductor element 5₁ corresponds to the helical profile of an imaginary line running purely axially in the untwisted state along the outside of the wire material 4 after being twisting.

In corresponding embodiments, the wire material 4 is a flat wire material 4_{F} with a polygonal cross section. FIGS. 1 to 3, 7 to 11 and 13 to 21 show associated exemplary embodiments in which the wire material 4 is specifically a flat wire material 4_{F} with a substantially rectangular cross section with two opposite broad sides 3_{B} and narrow sides 3_{S} in each case.

In corresponding implementations, the wire material 4 has a ratio of wire width Bp, i.e. maximum transverse dimension, to wire thickness D_{D}, i.e. minimum transverse dimension, of between 1:1 and 4:1. In the examples shown with the flat wire material 4_{F} with a rectangular cross section, this ratio is in the range of a little over 1:1 to approximately 3:1. A ratio of 1:1 corresponds to a square wire cross section.

FIGS. 4 to 6 and 12 show exemplary embodiments in which the wire material 4 of the twisted core wire 3 is a round wire material 4_{R} with a substantially circular cross section, i.e. the ratio of wire width B_{D} to wire thickness D_{D} is 1:1.

In corresponding embodiments, the conductor element 5₁ is inserted into an associated conductor receiving groove 6 in the core wire 3. Exemplary embodiments of this kind are shown in FIGS. 4 to 6 and 12. In the example of FIGS. 4 and 5, the core wire 3 has a single conductor receiving groove 6 into which an associated conductor element 5₁ is inserted. In the examples of FIGS. 6 and 12, the core wire 3 has two diametrically opposite conductor receiving grooves 6 into each of which a conductor element 5₁, 5₂ of the conductor element arrangement 5 consisting in this case of the two conductor elements 5₁, 5₂ is inserted. It is understood that the respective conductor receiving groove 6 runs in a helical manner on the outer circumference of the core wire 3 in accordance with the twisting of the core wire 3, as can be seen from FIG. 4 for example.

In the exemplary embodiments of FIGS. 4 to 6 and 12, the conductor element 5₁, 5₂ inserted into the respective conductor receiving groove 6 terminates externally flush with the core wire 3, without projecting radially to the outside beyond this core wire. In alternative embodiments, provision may be made for at least one of the conductor elements 5₁, 5₂ to project radially to the outside beyond the core wire 3.

In the examples shown in FIGS. 4 to 6 and 12, the respective conductor receiving groove 6 is introduced into the round wire material 4_{R} of the core wire 3. In alternative embodiments not shown, the respective conductor receiving groove 6 of the core wire 3 is introduced into another wire material 4, for example into a flat wire material 4_{F} with a polygonal cross section, as is used specifically as a rectangular wire material 4 in the examples of FIGS. 1 to 3, 7 to 11 and 13 to 18.

In corresponding embodiments, the conductor element 5₁ is arranged on a broad side 3_{B} of the core wire 3. Associated exemplary embodiments are shown in FIGS. 1 to 3, 9 to 11 and 13 to 15. With this positioning, the conductor element 5₁ does not increase the lateral dimension of the twisted core wire 3 determined by the wire width B_{D} when using rectangular flat wire material 4_{F}. In the case of the rectangular flat wire material 4_{F}, the wire width B_{D} corresponds to the distance between the opposite narrow sides 3_{S} of the flat wire material 4_{F}.

In corresponding embodiments, the conductor element arrangement 5 contains a first conductor element 5₁ on a first broad side 3_{B} of the core wire 3 and a second conductor element 5₂ on a second broad side 3_{B} of the core wire 3. Associated exemplary embodiments are shown in FIGS. 3 and 9 to 11 and 18 to 21.

In corresponding embodiments, the core wire 3 and the conductor element arrangement 5 are embedded in an embedding material 7 from the proximal region 1 of the instrument to the distal region 2 thereof. In this case, the embedding material 7 forms a sheathing that embeds the core wire 3 and the conductor element arrangement 5 preferably without cavities. An associated exemplary embodiment is shown in FIG. 14. The embedding material 7 secures the conductor element arrangement 5 in its position on the twisted core wire 3. Embodiments with such an embedding material 7 can be produced, for example, by extrusion, this keeping the outlay on manufacturing low.

In the exemplary embodiments of FIGS. 13 and 14, the conductor element arrangement 5 comprises two conductor elements 5₁, 5₃, both of which are arranged on a common broad side 3_{B} of the core wire 3. In the case shown, the two conductor elements 5₁, 5₃ are arranged radially adjacent to each other.

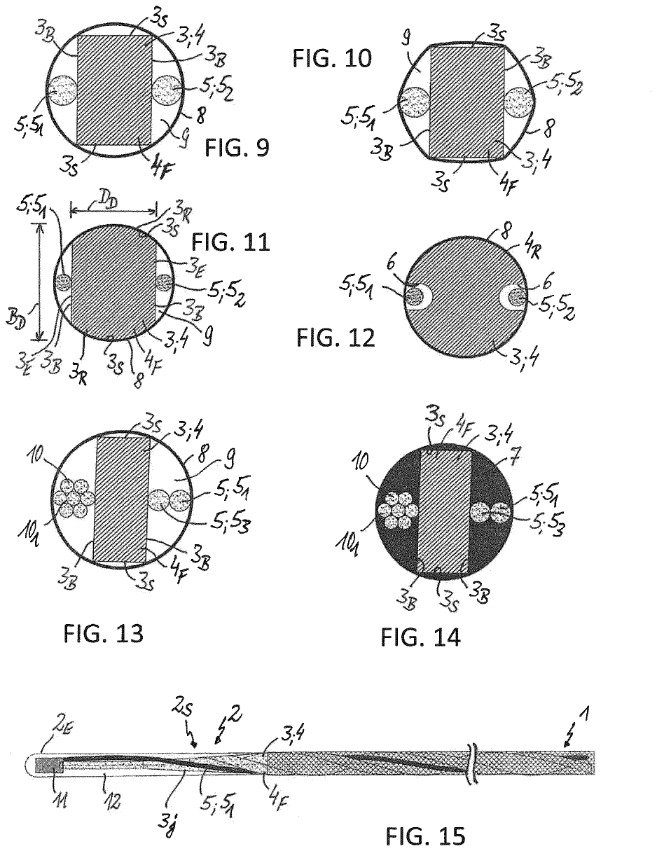

In corresponding embodiments, the instrument contains a sheathing tube 8 which surrounds the core wire 3 and the conductor element arrangement 5 from the proximal region 1 of the instrument to the distal region 2 thereof. Associated embodiments are shown in FIGS. 9 to 13 and 15 to 18. The sheathing tube 8 is preferably fixedly, i.e. immovably, connected to the core wire 3, for example by shrink-fitting or adhesive bonding, and can secure the conductor element arrangement 5 in its position on the twisted core wire 3 and fulfill further functions, such as improving the sliding properties of the instrument and electrically insulating the instrument radially to the outside.

In corresponding embodiments, a fluid guiding lumen 9 extending from the proximal region 1 of the instrument to the distal region 2 thereof is formed between the core wire 3 and the sheathing tube 8. Associated exemplary embodiments are shown in FIGS. 9 to 11, 13 and 16 and 17. The fluid guiding lumen 9 can be formed from one part or from several parts consisting of several individual lumen. A respective fluid, for example water, a drug-containing fluid, a tissue fluid or a working fluid, can be guided, through the fluid guiding lumen 9, from the proximal instrument region 1 to the distal instrument region 2 or from the distal instrument region 2 to the proximal instrument region 1, depending on requirements. In this application, it may be advantageous to choose a relatively large width/thickness ratio for the core wire 3 of more than 4:1 in order to achieve a relatively large cross section of the fluid guiding lumen 9 with a given lateral dimension of the instrument.

In corresponding embodiments, the instrument contains a stiffening wire arrangement 10 or additional conductor elements of the conductor element arrangement 5 which comprises at least one stiffening wire 10₁ or additional conductor element which extends from the proximal instrument region 1 to the distal instrument region 2, specifically like the respective conductor element 5₁ with a helical profile synchronous to the twisting of the core wire 3. Associated embodiments are shown in FIGS. 13 and 14. In these examples, the stiffening wire 10₁ or the at least one further conductor element of the conductor element arrangement 5 consists of a stranded wire which is formed from a central strand and six surrounding strands. During use as at least one further conductor element of the conductor element arrangement 5, all the strands can together form an associated, multi-core conductor element or each strand individually can form a separate respective conductor element, depending on the embodiment. It is understood that the stiffening wire 10₁ or the at least one further conductor element of the conductor element arrangement 5 can have any other desired conventional wire structure, depending on requirements.

In the examples of FIGS. 13 and 14, the stiffening wire 10₁ or the at least one further conductor element of the conductor element arrangement 5 is arranged on a broad side 3_{B} of the flat wire material 4_{F} used there, specifically on that broad side 3_{B} which faces away from the conductor elements 5₁ and 5₃ of the conductor element arrangement 5. The stiffening wire arrangement 10 allows a desired bending behavior to be set for the instrument in the corresponding instrument portion, if required.

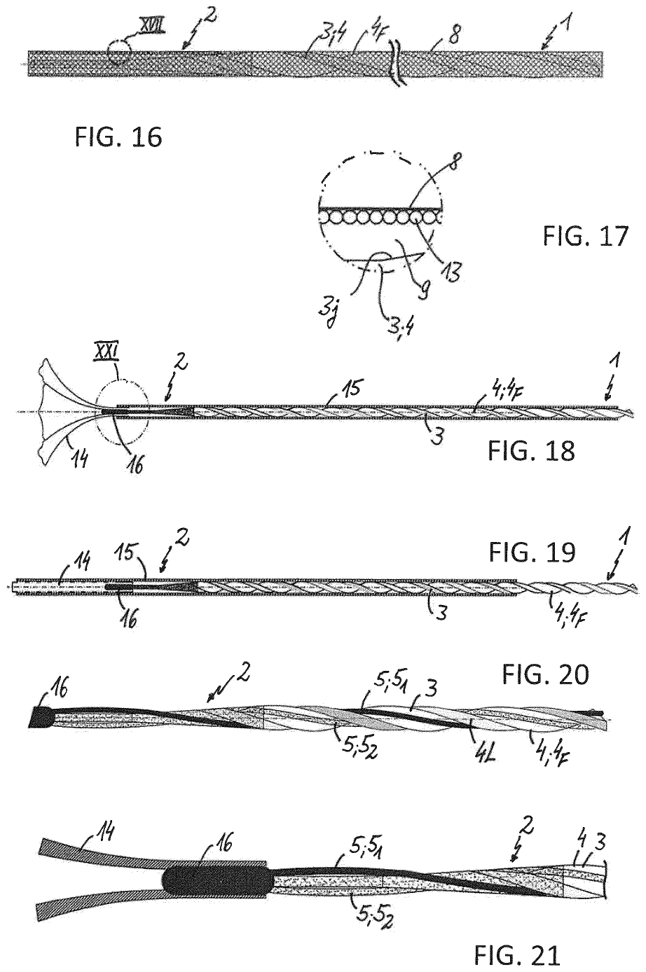

In corresponding embodiments, the core wire 3, in a distal tip region 2_{S} of the instrument, has a distal core wire portion 3j conically tapered to a round cross section. FIGS. 7, 8 and 15 to 21 show exemplary embodiments of this kind. In the guide wire exemplary embodiment of FIG. 15, an electrical functional element 11, to which the conductor element 5₁ is guided, is arranged at a distal end 2_{E} of the instrument. The distal tip region 2_{S} of the instrument has a distal sheathing 12 which surrounds the electrical functional element 11, the conically tapered distal core wire portion 3j and the part of the conductor element 5₁ running there. In the example shown, the sheathing 12 is formed from an embedding material in which the electrical functional element 11, the conically tapered core wire portion 3j and the conductor element 5₁ are embedded in this instrument portion. As an alternative, the distal sheathing 12 can also be implemented, for example, by a helical spring sheathing 13, as is used in the exemplary embodiments of FIGS. 8 and 16 and 17. In the guide wire exemplary embodiment of FIGS. 16 and 17, the sheathing tube 8 also extends in the distal tip region 2_{S} of the instrument or guide wire and acts there together with the helical spring sheathing 13 as a distal sheathing, the helical spring sheathing 13 being surrounded by the sheathing tube 8 radially to the outside.

In the embodiment of the instrument according to the invention as a snare instrument according to FIGS. 18 to 21, the core wire 3 in the implementation of FIG. 1 and more specifically of FIG. 7 is used by way of example, it being possible for one of the other core wire implementations mentioned to be used as an alternative. Furthermore, in this embodiment, the conductor element arrangement 5 contains, by way of example, the two conductor elements 5₁, 5₂ in the implementation according to FIG. 3 on the opposite broad sides 3_{B} of the core wire 3, as can be seen in FIG. 20. As an alternative, the conductor element arrangement 5 can be implemented in one of the other configurations explained above.

In this snare instrument, a snare basket 14, which is fixedly connected at its proximal end to the distal end of the core wire 3, for example by welding or adhesive bonding, distally adjoins the core wire 3 functioning as a pulling wire and is of any desired suitable design known per se, for example one of the designs as are mentioned in the prior art mentioned above, which therefore does not require any further explanation. Furthermore, the snare instrument in the usual manner contains an outer tube 15, in which the core wire 3 together with the adjacent conductor elements 5₁, 5₂ is axially movably guided and in which the snare basket 14 can be received at its distal end region in a folded-closed or folded-together fashion. By pushing the core wire 3 forward or pushing the outer tube 15 back, the snare basket 14 can be moved out of the outer hose 15 and unfolded or folded open. By pulling the core wire 3 back or pushing the outer tube 15 forward, the snare basket 14 can then be moved back into the outer tube 15 such that it is folded-closed or folded-together. FIGS. 18 and 19 show the instrument with the snare basket 14 folded-open or folded-together.

As can be seen from FIG. 21 in particular, a sensor element 16 is arranged in the distal end region of the instrument, which is preferably fixedly connected to the distal end of the core wire 3 and/or the proximal end of the snare basket 14, for example by adhesive bonding. The sensor element 16 fulfills one or more specified sensor functions, for example for identifying the position of the instrument in a tissue channel in the manner of a tracker or other conventional position determining means or for measuring one or more physical and/or chemical parameters in a tissue channel, such as temperature, pressure, composition of blood and/or surrounding tissue etc., or as an image sensor for obtaining optical images in the tissue channel. The conductor element arrangement 5 provides for the electrical and/or optical connection required by the sensor element 16 to be made with the proximal end region of the instrument, not of further interest here and therefore not shown, in which associated components for electrical and/or optical communication with the sensor element 16 are located. For this purpose, these components are suitably designed, depending on requirements, to supply, for example, the sensor element 16 with required sensor commands and/or electrical energy and/or light and/or to receive sensor signals emitted by the sensor element 16 and evaluate them.

In all the cases shown, the respective conductor element of the conductor element arrangement 5 is designed in an electrically conductive and/or light-guiding and/or fluid-conducting manner in line with the application. Accordingly, the conductor element in question consists of an electrically conductive material and/or of a light-guiding material and/or of a hollow material with a fluid-conducting cavity.

As is clear from the exemplary embodiments shown and the further exemplary embodiments explained above, the invention advantageously provides an elongate medical instrument, such as a guide wire or a snare instrument, with a twisted core wire, which can be manufactured relatively easily and has extended functionalities owing to the presence of the conductor element arrangement.

1.-13. (Canceled)

14. An elongate medical instrument, comprising:
a core wire extending from a proximal region of the instrument to a distal region of the instrument and formed from a wire material that is twisted around its longitudinal axis; and
a conductor element arrangement which comprises at least one element of the group comprised of electrically conductive elements, light-guiding elements and fluid-conducting conductor elements, said at least one element extending from the proximal region of the instrument to the distal region of the instrument in a helical manner synchronously with the twisting of the core wire.

15. The elongate medical instrument as claimed in claim 14, wherein the wire material is a flat wire material with a polygonal cross section.

16. The elongate medical instrument as claimed in claim 14, wherein the wire material has a ratio of wire width to wire thickness of between 1:1 and 4:1.

17. The elongate medical instrument as claimed in claim 14, wherein the conductor element is inserted into an associated conductor receiving groove of the core wire.

18. The elongate medical instrument as claimed in claim 14, wherein the conductor element is arranged on a broad side of the core wire.

19. The elongate medical instrument as claimed in claim 18, wherein the conductor element arrangement comprises a first conductor element on a first broad side of the core wire and a second conductor element on a second broad side of the core wire.

20. The elongate medical instrument as claimed in claim 14, wherein the core wire has at least one flat side surface and at least one rounded side surface.

21. The elongate medical instrument as claimed in claim 14, wherein the core wire and the conductor element arrangement are embedded in an embedding material from the proximal region of the instrument to the distal region of the instrument.

22. The elongate medical instrument as claimed in claim 14, further comprising:
a sheathing tube which surrounds the core wire and the conductor element arrangement from the proximal region of the instrument to the distal region of the instrument.

23. The elongate medical instrument as claimed in claim 22, wherein a fluid guiding lumen extending from the proximal region of the instrument to the distal region of the instrument is formed between the core wire and the sheathing tube.

24. The elongate medical instrument as claimed in claim 14, further comprising:
a stiffening wire arrangement which contains at least one stiffening wire which extends from the proximal region of the instrument to the distal region of the instrument in a helical manner synchronously with the twisting of the core wire.

25. The elongate medical instrument as claimed in claim 14, wherein
the core wire, in a distal tip region of the instrument, has a distal core wire portion conically tapered to a round cross section,
an electrical functional element, to which the conductor element is guided, is arranged at a distal end of the instrument, and
the distal tip region of the instrument has a distal sheathing which surrounds the electrical functional element, the conically tapered distal core wire portion and the part of the conductor element running there.

26. The elongate medical instrument as claimed in claim 14, wherein a sensor element is arranged in the distal region of the instrument and the conductor element arrangement is led with a distal end to the sensor element.

27. The elongate medical instrument as claimed in claim 14, said elongate medical instrument being a guide wire instrument or a snare instrument.
